(19) United States
(12) Patent Application Publication
(10) Pub. No.: US 2024/0416094 A1
(43) Pub. Date: Dec. 19, 2024

(54) DILATION CATHETER WITH INFLATABLE TIP FEATURE

(71) Applicant: Biosense Webster (Israel) Ltd., Yokneam (IL)

(72) Inventor: Cesar Fuentes-Ortega, Pasadena, CA (US)

(21) Appl. No.: 18/661,886

(22) Filed: May 13, 2024

Related U.S. Application Data
(60) Provisional application No. 63/472,642, filed on Jun. 13, 2023.

(51) Int. Cl. A61M 29/02; A61M 25/10

(52) U.S. Cl. CPC A61M 29/02; A61M 25/10

(57) ABSTRACT

A catheter system includes a balloon dilation catheter having a proximal end portion and a distal end portion. The balloon dilation catheter includes a dilation balloon lumen that is configured to couple with a first fluid supply. An expandable dilation balloon is fluidly connected to the dilation balloon lumen. The expandable dilation balloon is configured to transition between an inflated state and a non-inflated state. An expandable stop element is configured to selectively transition among a range of expanded states including a non-expanded state, a first expanded state, and a second expanded state. The expandable stop element defines an outer diameter such that the outer diameter defines a minimum diameter when the expandable stop element is in the non-expanded state and a maximum diameter when the expandable stop element is in the second expanded state.

### PRIORITY

This application claims priority to U.S. Provisional Pat. App. No. 63/472,642, entitled “Dilation Catheter with Inflatable Tip Feature,” filed Jun. 13, 2023, the disclosure of which is incorporated by reference herein, in its entirety.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pat. No. 11,964,114, entitled “Shaft Deflection Control Assembly for ENT Guide Instrument,” issued Apr. 23, 2024, the disclosure of which is incorporated by reference herein, in its entirety.

In the context of Eustachian tube dilation, a dilation catheter or other dilation instrument may be inserted into the Eustachian tube and then be inflated or otherwise expanded to thereby dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Pat. No. 11,013,896, entitled “Method and System for Eustachian Tube Dilation,” issued May 25, 2021, the disclosure of which is incorporated by reference herein, in its entirety.

It may be desirable to provide instrumentation with the ability to change and adjust based on the anatomical needs of the patient. While several systems and methods have been made and used to dilate an anatomical passageway, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an example of a guide instrument for use in ear, nose, and throat (ENT) procedures, with a guide slider and an instrument slider each in respective proximal positions;

FIG. 2 depicts a perspective view of the instrument of FIG. 1, with the guide slider in a distal position and a dilation catheter advanced distally, and with the guide shaft in a longitudinally extended state;

FIG. 3A depicts an enlarged side view of the distal end of the dilation catheter of the dilation catheter system of FIG. 1, with a dilator of the dilation catheter in an inflated state and a tip of the dilation catheter in a deflated state;

FIG. 3B depicts an enlarged side view of the distal end of the dilation catheter of the dilation catheter system of FIG. 1, with the dilator of the dilation catheter in an inflated state and the tip of the dilation catheter in a first inflated state;

FIG. 3C depicts an enlarged side view of the distal end of the dilation catheter of the dilation catheter system of FIG. 1, with the dilator in the inflated state and the tip in a second inflated state;

FIG. 4 depicts a schematic view of a first example of a dilation catheter system;

FIG. 5 depicts a schematic view of a second example of a dilation catheter system;

FIG. 6 depicts a schematic view of a third example of a dilation catheter system;

FIG. 7A depicts a cross-sectional view of a human ear showing the inner, middle and outer ear portions and the Eustachian tube connecting the middle ear with the nasopharynx region of the throat, with the tip balloon of the dilation instrument system of FIG. 1 being sized with a distal tip gauge;

FIG. 7B depicts a cross-sectional view of the human ear of FIG. 7A, with the dilation catheter of FIG. 2 inserted in the Eustachian tube while the tip balloon of the dilation catheter is in an inflated state and the dilation balloon is in a deflated state;

FIG. 7C depicts a cross-sectional view of the human ear of FIG. 7A, with the dilation catheter of FIG. 2 inserted in the Eustachian tube, with the tip balloon of the dilation catheter in the inflated state, and with the dilation balloon of the dilation catheter being inflated to dilate the Eustachian tube;

FIG. 7D depicts a cross-sectional view of the human ear of FIG. 7A, with the dilation catheter of FIG. 2 removed from the Eustachian tube while the tip balloon and dilation balloon of the dilation catheter are both in the deflated state;

FIG. 8 depicts a cross-sectional view of the human ear of FIG. 7A, with the dilation catheter of FIG. 2 being removed from the Eustachian tube while the tip balloon and the dilation balloon of the dilation catheter are both in the deflated state; and

FIG. 9 depicts a cross-sectional view of the human ear of FIG. 7A, with the dilation catheter of FIG. 2 being inflated and sized while in the Eustachian tube.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Example of an ENT Guide Instrument

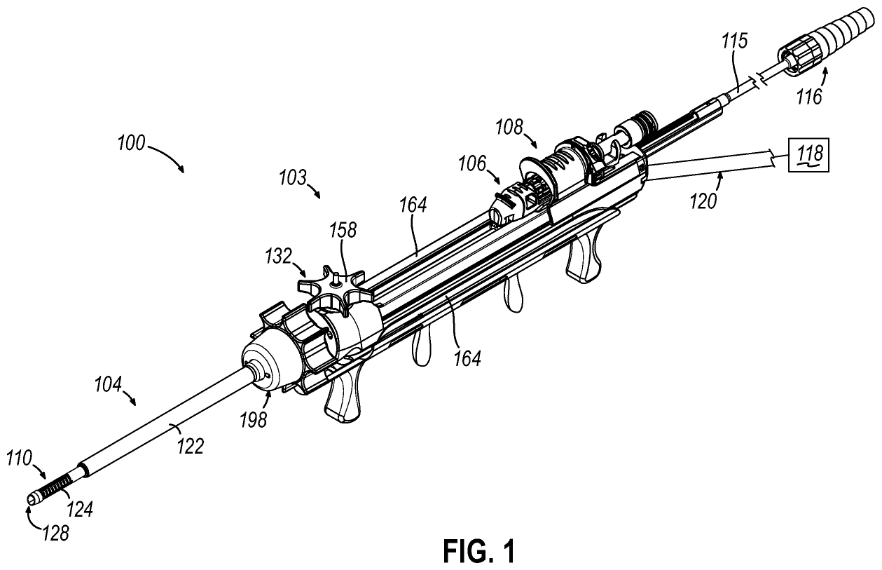

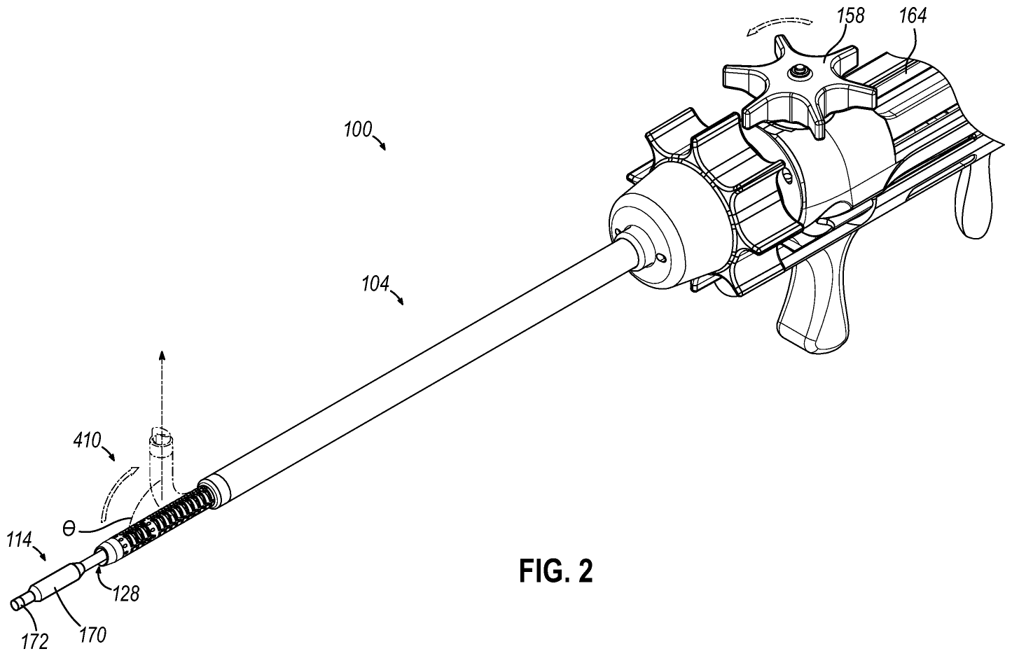

FIGS. 1-2 show an ENT guide instrument (100) that may be used for medical procedures within the ear, nose, or throat. Such procedures may include ablation within the nasal cavity; dilation of an ostium of a paranasal sinus, a Eustachian tube, or some other anatomical passageway (e.g., within the ear, nose, or throat, etc.); suctioning of fluids or debris from within the nasal cavity or ear; performing a biopsy within an ear, nose, or throat; removing bone or tissue from within the nasal cavity; or any other suitable procedure within the ear, nose, or throat as will be apparent to those skilled in the art in view of the teachings herein. ENT guide instrument (100) of the present example provides adjustability that enables the operator to use ENT guide instrument (100) in different scenarios, without requiring the operator to switch between different instruments. For instance, ENT guide instrument (100) may be used to access various different anatomical passageways by making simple adjustments to structural features of the instrument; and then address those different anatomical passageways with various ENT instrumentation that is coupled with ENT guide instrument (100).

ENT guide instrument (100) of this example includes a handle assembly (103) having a housing (164), a guide shaft assembly (104) extending distally from handle assembly (103); a guide shaft actuation assembly, or guide slider (106), which is slidably coupled with handle assembly (103); and an instrument slider (108) slidably coupled with handle assembly (103). One or more instruments such as balloon dilation catheter (114) may be inserted into guide shaft assembly (104) of ENT guide instrument (100) via port (116) and through inner shaft (not shown), which spans through handle assembly (103). In the present example, the inner shaft is coaxially disposed within guide shaft assembly (104), and balloon dilation catheter (114) is coaxially disposed within the inner shaft through use of dilation catheter shaft (115). A suction source (118) is coupled with guide shaft assembly (104) of ENT guide instrument (100) via a suction port (120) and a conduit (not shown) which spans through handle assembly (103). In some versions, suction port (120) is omitted.

Handle assembly (103) is sized and configured to be grasped and operated by a single hand of an operator. The operator may selectively operate guide slider (106) or instrument slider (108), with the same single hand that grasps handle assembly (103). The operator may translate guide slider (106) distally along handle assembly (103) to thereby telescopically translate guide shaft assembly (104) such that the distal end (110) of guide shaft assembly (104) longitudinally translates distally away from handle assembly (103). The operator may also translate guide slider (106) proximally along handle assembly (103) to thereby telescopically translate guide shaft assembly (104) such that the distal end (110) of guide shaft assembly (104) longitudinally retracts proximally toward handle assembly (103). Thus, guide shaft assembly (104) may be effectively longitudinally lengthened or shortened during operation, as needed, prior to or during the operation.

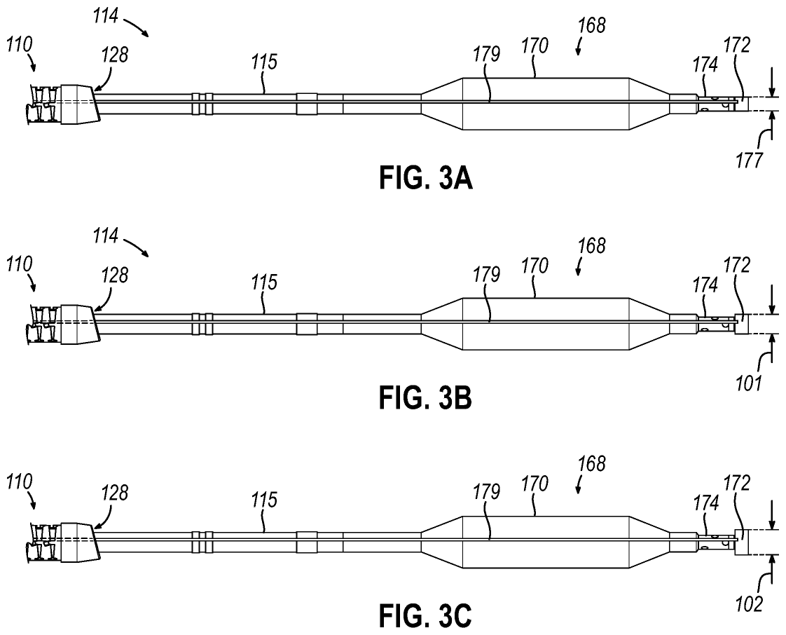

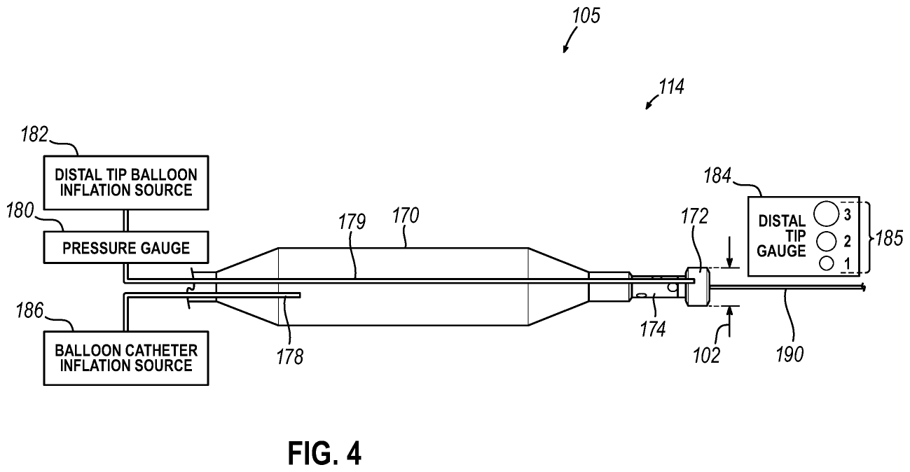

An operator may advance instrument slider (108) distally along handle assembly (103) to thereby advance a balloon dilation catheter (114) distally through the inner shaft and guide shaft assembly (104), such that the distal tip of balloon dilation catheter (114) is translated through an opening (128) and distal to the distal end of guide shaft assembly (104). With balloon dilation catheter (114) advanced to a distal position, the operator may then utilize balloon dilation catheter (114) within an anatomical passageway in which balloon dilation catheter (114) is positioned. Various examples of balloon dilation catheters are described herein with respect to FIG. 3A to FIG. 6; while other examples will be apparent to those skilled in the art in view of the teachings herein. In some versions, a guidewire (190), shown on FIG. 4, is included along with balloon dilation catheter (114). Alternatively, guidewire (190) may be omitted.

As is illustrated in FIG. 2, rotation of thumbwheel (158) causes longitudinal translation of a push-pull wire (not shown) within guide shaft assembly (104), and longitudinal translation of the push-pull wire thereby causes straightening or bending of a flex section of guide shaft assembly (104) to achieve a desired bend angle (0). This may be useful for further guiding balloon dilation catheter (114). By way of example only, such steering features of ENT guide instrument (100) (among other features of ENT guide instrument (100)) may be configured in accordance with at least some of the teachings of U.S. Patent Pub. No. 2021/0386274, entitled “ENT Guide with Advanceable Instrument and Advanceable Endoscope Shaft,” published on Dec. 16, 2021, the disclosure of which is incorporated by reference herein, in its entirety.

II. Example of Dilation Catheter with Partially Inflatable Tip Balloon

In some circumstances, it may be beneficial for a single balloon dilation catheter (114) to be capable of accommodating several different patient anatomies such that an operator may select a single balloon catheter without having to choose from several differently configured balloon catheters based on patient size or anatomy. Having a single balloon dilation catheter (114) that is “universal” among different patient anatomies may reduce the need to purchase and store excess balloon dilation catheters (114). A balloon dilation catheter (114) having a tip balloon (172) that is configured for selectively varying an outer diameter by partially inflating the tip balloon (172) may allow an operator to accommodate a wide range of patient sizes or anatomies. The balloon dilation catheter (114) described below may be capable of accommodating these needs through use of the above-mentioned ENT guide instrument (100) or with any other suitable kind of guide instrument.

FIG. 3A shows distal end of balloon dilation catheter (114) having tip balloon (172) in a deflated state, whereas FIG. 3B shows the tip balloon (172) in a partially inflated state having a greater diameter (101) than outer shaft diameter (177) and after having received the fluid from tip lumen (179). Tip balloon (172) may be resiliently biased toward the deflated state through the material forming tip balloon (172) itself, such as by using an extensible material; it may be imparted by a resilient sleeve disposed about a non-extensible material forming tip balloon (172); or it could be otherwise imparted. Additionally, other versions may lack the resilient bias toward the deflated state. A version lacking the resilient bias toward the deflated state may, for example, include an applied vacuum that transitions tip balloon (172) into the deflated state.

As discussed in greater detail below, tip lumen (179) extends through the dilation balloon (170) and to the inflatable tip balloon (172) positioned on the distal shaft end (174). Distal shaft end (174) defines the outer shaft diameter (177). The tip balloon (172) in the deflated state defines a deflated outer diameter generally equivalent to the outer shaft outer diameter (177) for insertion through relatively tight spaces (e.g., paranasal sinus ostia) within the patient. Upon receiving the fluid for inflation, tip balloon (172) projects radially outwardly from the distal shaft end (174) to define an inflated outer diameter (102) that is larger than the outer shaft diameter for preventing the balloon dilation catheter (114) from advancing to an undesirable position within the patient, such as beyond the isthmus (29). While the tip balloon (172) is shown in FIGS. 3B-3C as inflating to a generally cylindrical configuration, it will be appreciated that the tip balloon (172) may be configured to inflate to any desirable shape, such as a blueberry shape. Thus, the tip balloon (172) is not intended to be unnecessarily limited to the shape shown and described herein.

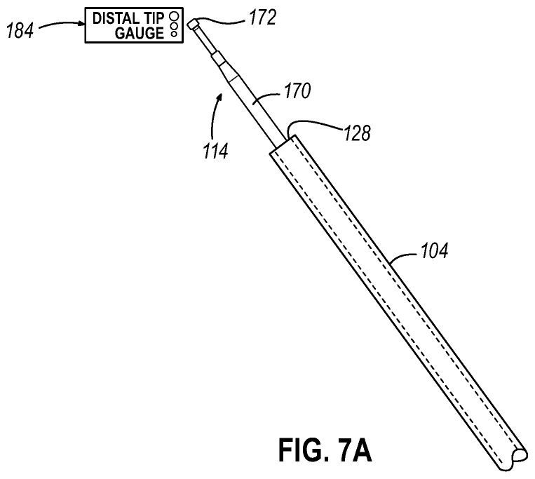

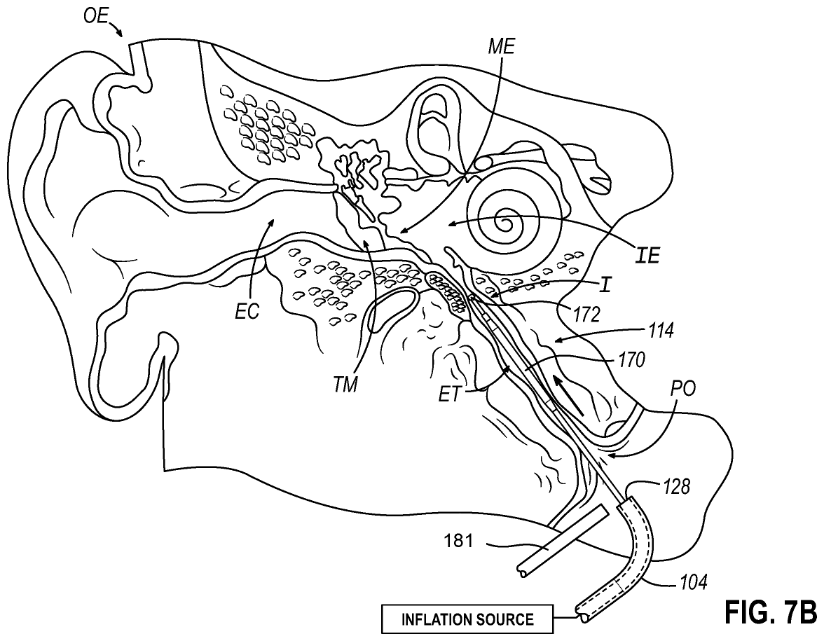

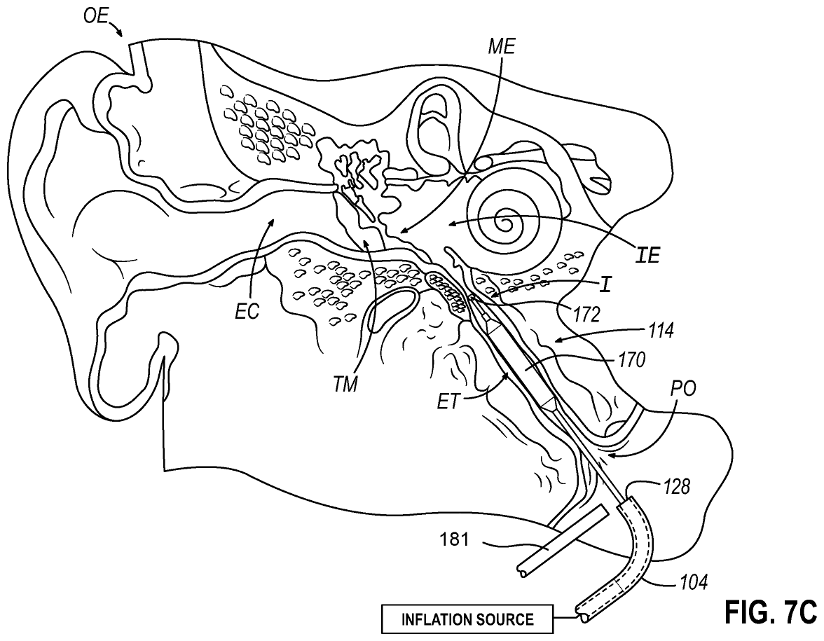

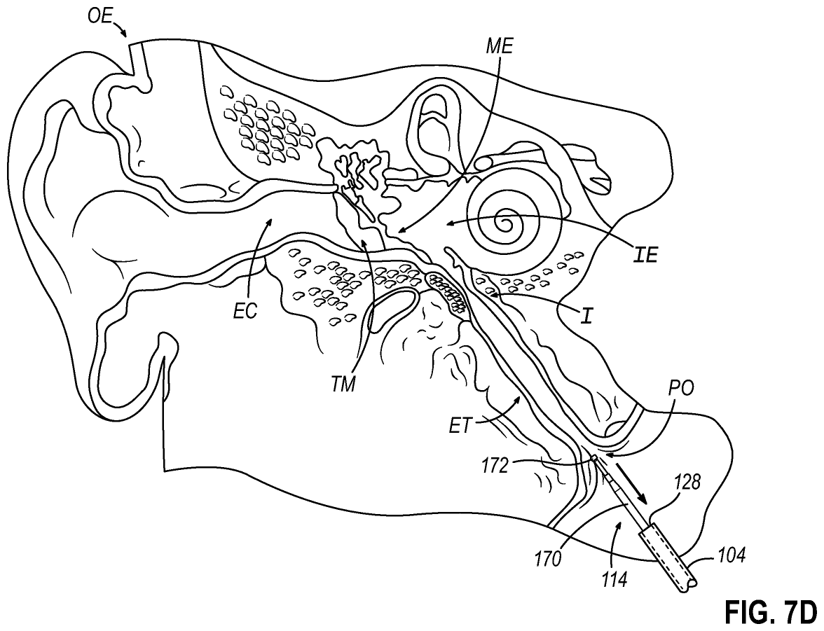

Tip balloon (172) may be capable of inflating to any diameter between the fully deflated state shown in FIG. 3A, where diameter of tip balloon matches or is less than outer shaft diameter (177), to the fully inflated state shown in FIG. 3C having inflated outer diameter (102). As shown in FIG. 3B, tip balloon (172) may be capable maintaining a partial inflation between that which is shown in FIGS. 3A and 3C. Partial inflation of tip balloon (172) may be beneficial for patients with small anatomical passages who may not require a fully inflated tip balloon (172) in order to perform the procedure. An example of this may be pediatric patients with smaller anatomies; and particularly a smaller isthmus (I) as described below with reference to FIG. 7B. As an example only and in no way intended to be limiting, when partially inflated, tip balloon (172) may include a partially inflated diameter (101) that is greater than the outer shaft diameter but less than approximately 0.08 inches.

The diameter of tip balloon (172) at a given moment may be determined through visual observation of a property of tip balloon (172) that changes with diameter. For instance, in the deflated state shown in FIG. 3A, tip balloon (172) may be largely opaque. As tip balloon (172) expands from the deflated state of FIG. 3A to the fully inflated state of FIG. 3B, tip balloon (172) may become increasingly translucent. An operator may thus be able to visually gauge the diameter based on the degree of translucency.

As another example, in versions where tip balloon (172) comprises an extensible material, tip balloon (172) material may be capable of changing colors as it is stretched such that a particular observed color indicates a particular diameter. When the operator is able to observe a particular color, they can refer to a color guide which indicates the current diameter of tip balloon (172).

Tip balloon (172) may comprise a non-extensible material such that, once fully inflated, tip balloon (172) may not increase further in diameter with an increased fluid pressure. With a non-extensible material, tip balloon (172) may still be capable of maintaining a consistent diameter whether partially or fully inflated. Tip balloon (172) may also be made with an extensible material such that the diameter of tip balloon (172) may increase as pressure increases. Tip balloon (172) may also comprise a combination of extensible and non-extensible layers. In such a combination, the extensible layer may provide a resilient bias toward the deflated configuration while the non-extensible layer restricts the size to which the tip balloon (172) may be inflated.

III. Examples of Systems and Techniques for Operating Dilation Catheter with Partially Inflatable Tip Balloon

A system for dilating dilation balloon (170) and tip balloon (172) as desired by the operator may be useful in allowing the operator to advance balloon dilation catheter (114) appropriately into the inner ear of the patient. Additionally, the operator may find added value in being able to confirm the diameter to which tip balloon (172) has been inflated. The following description provides several examples of systems and methods that may be used to control operation of dilation catheter (114).

### A. Example of First System for Partial Inflation of Tip Balloon

FIG. 4 shows an example of a system (105) for inflating both dilation balloon (170) and tip balloon (172). Tip lumen (179) may extend along a length of balloon dilation catheter (114) such that a distal end of tip lumen (179) is in fluid communication with tip balloon (172). Extending proximally through balloon dilation catheter (114), a proximal end of tip lumen (179) may be in fluid communication with an optional pressure gauge (180) and a distal tip balloon inflation source (182).

Distal tip balloon inflation source (182) may be operable to selectively inflate and deflate tip balloon (172) (e.g., with saline, etc.) to any diameter between and including the deflated configuration shown in FIG. 3A and the inflated configuration shown in FIG. 3C. Distal tip balloon inflation source (182) may be positioned external to ENT guide instrument (100) and may be capable of pressurizing tip balloon (172) to a selectable pressure and thus a specific diameter.

Pressure gauge (180) may be included on or separate from ENT guide instrument (100) and may be operable to convey tip lumen (179) pressure to operator. Pressure gauge (180) may include pressure readings to operator and may also include diameter indicators of tip balloon (172). In other words, when pressure gauge (180) senses a particular pressure in tip lumen (179), pressure gauge (180) may be capable of conveying to the operator the tip balloon (172) diameter related to that particular pressure. Pressure gauge (180) may be analog or digital.

A distal tip gauge (184) may be provided with ENT guide instrument (100) to indicate to the operator the tip balloon (172) diameter. Distal tip gauge (184) may be a flat member including a series of holes, each hole having a gradually increased diameter from the last and an indicia (185) adjacent to the hole to indicate the diameter. The operator may use distal tip gauge (184) to act as a go/no-go gauge. This is to say that once tip balloon (172) is inflated, the operator may confirm the desired diameter by placing tip balloon (172) through a first hole of distal tip gauge (184) and then confirming that tip balloon (172) is then obstructed by a smaller second hole of distal tip gauge (184). By doing this, the operator may confirm that inflated outer diameter (102) of tip balloon (172) is sized between the first and second holes of distal tip gauge (184). Each indicia (185) may indicate the diameter of the hole and may also indicate the patient population with which that diameter is indicated for. For instance, a large diameter hole may be indicated for an adult population whereas a smaller diameter hole may be indicated for a child population.

To inflate dilation balloon (170), a dilation lumen (178) may include a distal end in fluid communication with dilation balloon (170) and a proximal end in fluid communication with balloon catheter inflation source (186). Dilation lumen (178) may extend from dilation balloon (170) to balloon catheter inflation source (186). Distal tip balloon inflation source (182) and balloon catheter inflation source (186) may each be in the form of a syringe or anything capable of providing the needed fluid pressure to inflate balloons (170, 172). Distal tip balloon inflation source (182) and balloon catheter inflation source (186) may also be fluidly isolated from each other and be operated independently of each other in this example. As a result, dilation balloon (170) and tip balloon (172) may each have a dedicated inflation source (186, 182), respectively. However, dilation balloon (170) and tip balloon (172) may share the same inflation source in other versions, as will be described in greater detail below.

### B. Example of Second System for Partial Inflation of Tip Balloon

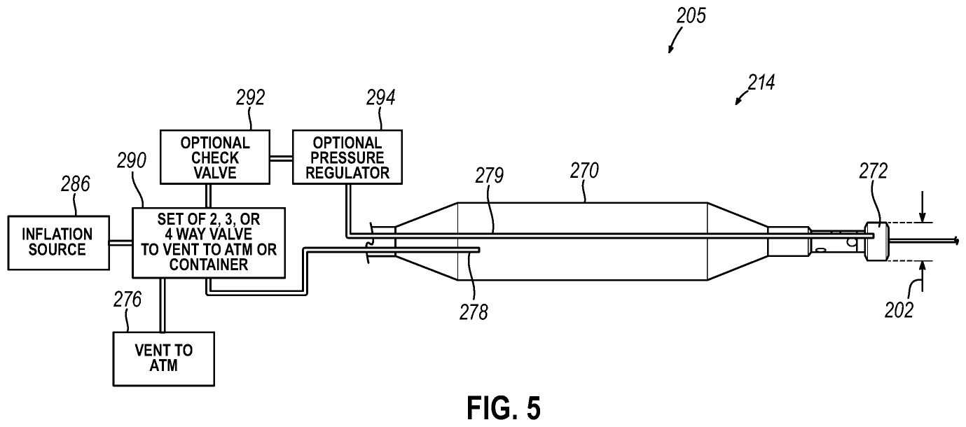

In some circumstances, it may be desirable to set the inflated outer diameter of tip balloon (172) to a pre-determined diameter. This may be useful in setting or changing the diameter while tip balloon (172) is in the Eustachian tube (ET). An example of an arrangement providing such functionality is shown in FIG. 5. In particular, FIG. 5 shows an example of a system (205) including a balloon dilation catheter (214), which is substantially similar to balloon dilation catheter (114) except for the differences described below. System (205) of this example also includes inflation source (286), valve (290), check valve (292), and pressure regulator (294).

Valve (290) may comprise any of either a 2, 3, or 4 way valve or a series of valves and may selectively provide fluid from inflation source (286) to either or both dilation lumen (278) and/or tip lumen (279). When valve (290) is in a closed position, fluid from inflation source (286) is prohibited from reaching dilation balloon (270) and/or tip balloon (272). When valve (290) is in an open position, fluid from inflation source (286) may selectively travel from inflation source (286) to dilation balloon (270) and/or tip balloon (272). Valve (290) may also include a vent or suction position capable of selectively venting dilation balloon (270) and/or tip balloon (272) to atmosphere through vent lumen (276) to thereby deflate dilation balloon (270) and/or tip balloon (272). Valve (290) may also be capable of backflowing fluid from dilation balloon (270) and/or tip balloon (272) to inflation source (286) should the pressure be lower at inflation source (286) than in balloons (270, 272). Valve (290) may also be capable of equalizing pressures between balloons (270, 272). Valve (290) may be manually operated or electrically operated and may be positioned on or distal to ENT guide instrument (100).

Positioned between valve (290) and tip balloon (272) may be a check valve (292). Check valve (292) may be optional and is operable to prevent fluid flowing from tip balloon (272) towards valve (290). Check valve (292) may be included to ensure inflated outer diameter (102) of tip balloon (272) does not decrease in diameter during a dilation procedure. It may also be included to help ensure a one-time use operation of ENT guide instrument (100) or to indicate to a potential operator that the instrument has been previously used and therefore should not be used again.

Positioned between valve (290) and tip balloon (272) may be a pressure regulator (294). Shown distal to check valve (292), pressure regulator (294) may be on either side of check valve (292). Pressure regulator (294) may control the maximum fluid pressure at tip balloon (272) to thereby control inflated outer diameter (202) of tip balloon (272). Pressure regulator (294) works to limit the pressure seen at tip balloon (272) and can be adjustable by the operator. Pressure regulator (294) may include indicia capable of relaying to an operator the preselected maximum pressure to be seen at tip balloon (272) and also the inflated outer diameter (202) that would result from the preselected pressure. Indicia may also include the anticipated patient population for which inflated outer diameter (202) is indicated for. Pressure regulator (294) may also include a pressure gauge to indicate either both or one of the current line pressures from valve (290) and/or the pressure in tip lumen (279) from tip balloon (272).

### C. Example of Third System for Partial Inflation of Tip Balloon

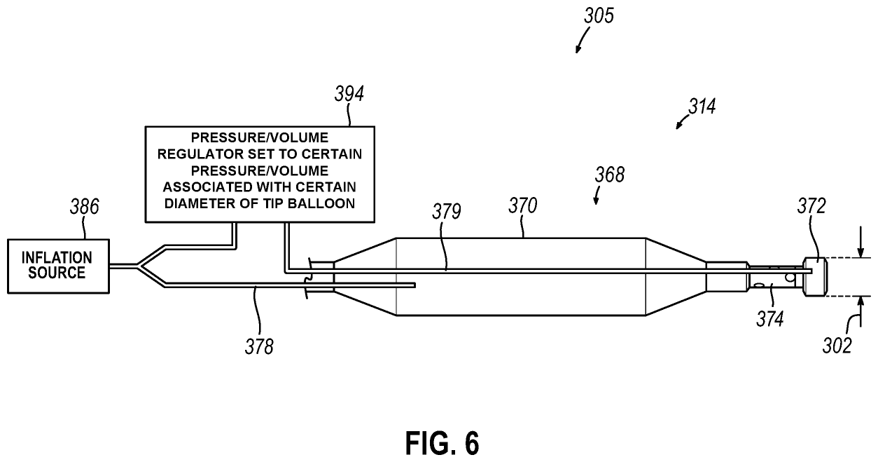

In some circumstances, it may be desirable to easily provide at least some fluid pressure to both balloons (170, 172) simultaneously. FIG. 6 shows an arrangement that is capable of providing such functionality. In particular, FIG. 6 shows an example of a system (305) including a balloon dilation catheter (314) having a tip balloon (372), tip lumen (379), dilation balloon (370), dilation lumen (378), and optional pressure and/or volume regulator (394). Balloon dilation catheter (314) is substantially similar to balloon dilation catheters (114, 214), except for the differences described below. Tip lumen (379), dilation lumen (378), and pressure regulator (394) may be in fluid communication with inflation source (386) such that inflation source (386) is operable to pressurize them. Tip lumen (379) may include pressure regulator (394) which operates in the same manner as pressure regulator (294) to limit the maximum pressure seen at tip balloon (372) while also indicating to an operator the current pressure of both lumens (378, 379). Tip lumen (379) may additionally or alternatively include volume regulator (394), which operates to limit the maximum inflation volume provided at tip balloon (372), while also indicating to an operator the current pressure and/or volume at both lumens (378, 379). Pressure/volume regulator (394) may be positioned along or proximal to ENT guide instrument (100). A volume regulator may be positioned with or in lieu of a pressure regulator in any of the systems described herein.

The pressure required to fully inflate tip balloon (372) may be lower than the pressure required to dilate Eustachian tube (ET) when using dilation balloon (370). Additionally, the pressure required to fully inflate tip balloon (372) may be lower than the pressure required to inflate dilation balloon (370) by any amount. In other words, tip balloon (372) may be fully inflated at a given pressure before dilation balloon (370) begins to inflate. In this configuration, an operator may set pressure regulator (394) to achieve the desired outer diameter (302) of tip balloon (372) and then, using inflation source (386), inflate tip balloon (372) to that diameter without inflating dilation balloon (370). Then, once balloon dilation catheter is appropriately positioned in the Eustachian tube (ET), operator may increase the fluid pressure from inflation source (386) to thereby dilate the Eustachian tube (ET) without changing outer diameter (302) of tip balloon (372). Tip balloon (372) may be capable of withstanding/receiving the same maximum pressure as seen by dilation balloon (370) during dilation.

### D. Example of First Method of Use for Partially Inflatable Tip Balloon

FIGS. 7A-9 show methods of using the previously described systems (105, 205, 305). While components of system (105) are shown in FIGS. 7-9, it is understood that systems (205, 305) may also be used in a similar method. In scenarios where the Eustachian tube (ET) remains persistently blocked or fails to open when it should, it may be desirable to dilate the Eustachian tube (ET). As shown in FIG. 7A, an operator may first inflate a tip balloon (172) to an appropriate size for the patient by using a distal tip gauge (184), a pressure regulator, injecting a known volume, or through visual observation of a property of the tip balloon (172). Distal tip gauge (184) may provide differently-sized openings to correspond with differently sized patients, such as a relatively small diameter opening for pediatric patient and a relatively large opening for an adult patient. In some scenarios, the operator inserts tip balloon (172) into a selected opening in distal tip gauge (184) while tip balloon (172) is in a deflated state; then inflates tip balloon (172) until tip balloon (172) reaches a diameter approximately the same size as the diameter of the selected opening in distal tip gauge (184). Tip balloon (172) may include a smaller diameter when fully inflated than distal opening (128) of guide shaft assembly (104) such that dilation balloon (114) is capable of fully retracting into guide shaft assembly (104).

FIG. 7B shows anatomical structures associated with a patient's ear, including the outer ear (OE), the ear canal (EC), the tympanic membrane (TM), the middle ear (ME), and the Eustachian tube (ET). The Eustachian tube (ET) is in fluid communication with the nasopharynx via the pharyngeal ostium (PO). The isthmus (I) is a narrowed region (e.g., approximately 1 mm in diameter) between the Eustachian tube (ET) and the middle ear (ME). The Eustachian tube (ET) provides a pathway for ventilation from the nasopharynx to the middle ear (ME); and for drainage from the middle ear (ME) to the nasopharynx. When functioning properly, the Eustachian tube (ET) may open for a fraction of a second periodically in response to swallowing or yawning. In so doing, it allows air into the middle ear (ME) to replace air that has been absorbed by the middle ear lining (mucous membrane) or to equalize pressure changes occurring on altitude changes. In instances where fluid is built up in the middle ear (ME), an opened Eustachian tube (ET) also provides a pathway for such fluid to drain from the middle ear (ME) into the nasopharynx.

As shown in FIG. 7B, with tip balloon (172) appropriately sized, the operator may begin the operation by inserting guide catheter (114) through a patient's nose or mouth and positioning guide shaft assembly (104) in the nasopharynx such that distal opening (128) is oriented toward the pharyngeal ostium (PO). This may be performed under visualization from an endoscope (181), if desired. Dilation catheter (114) may be advanced through guide shaft assembly (104) to position tip balloon (172) within the Eustachian tube (ET) and against the isthmus (I). In some cases, the operator may feel the resistance imparted by the isthmus (I) on tip balloon (172), and thereby confirm proper placement of dilation balloon (170) within the Eustachian tube (ET) through tactile feedback. As shown in FIG. 7C, once tip balloon (172) has contacted and been obstructed by isthmus (I) within the Eustachian tube (ET), dilation balloon (170) may then be inflated to thereby dilate the Eustachian tube (ET).

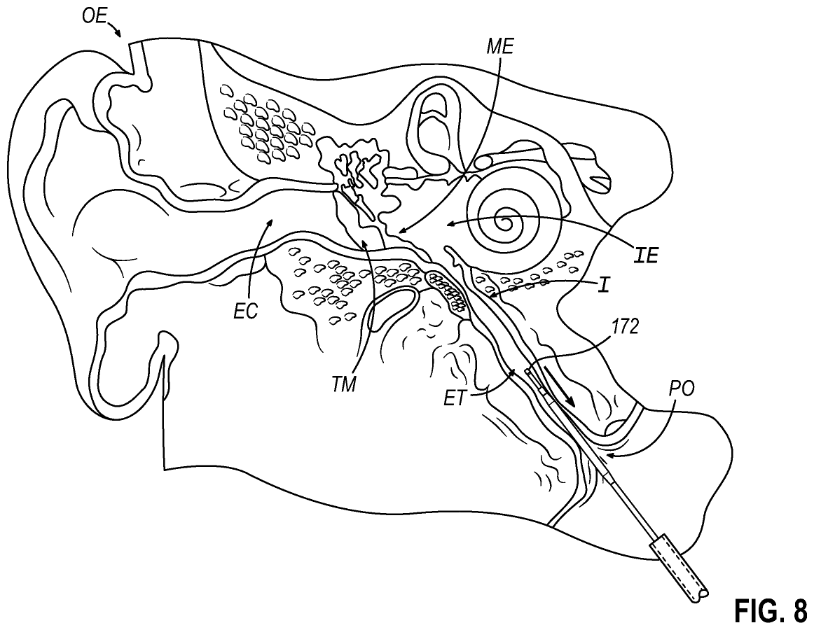

Once the Eustachian tube (ET) has been dilated, the operator may deflate dilation balloon (170) (and, optionally, tip balloon (172)) to decrease the effective diameter of dilation catheter (114) and thereby allow for retraction of dilation catheter (114) from the patient. As shown in FIG. 7D, tip balloon (172) may remain partially or fully inflated while dilation balloon (170) is fully deflated. To deflate either dilation balloon (170) or tip balloon (172), an operator may use any of the prescribed systems (105, 205, 305). The operator may deflate balloons (170, 172) through use of a valve, a vent, a pressure regulator, or applying suction from a vacuum source. As shown in FIG. 8, tip balloon (172) may be used in substantially the same method as tip balloon (172) depicted in the FIG. 7 series but may be fully deflated prior to retraction (i.e., between the state of operation shown in FIG. 7C and the state of operation shown in FIG. 7D).

### E. Example of Second Method of Use for Partially Inflatable Tip Balloon

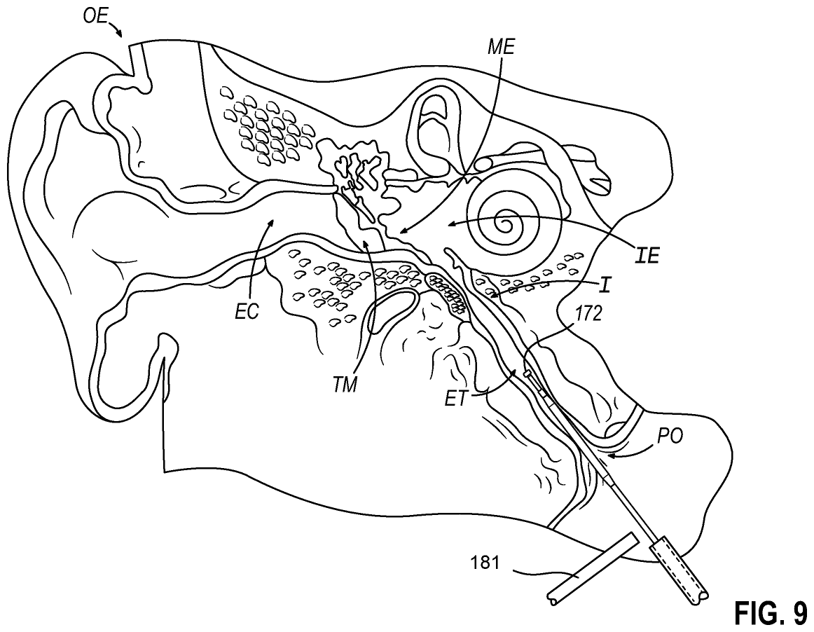

In some circumstances it may be desirable to expand tip balloon (172) after initial insertion of tip balloon (172) in the Eustachian tube (ET); but before full insertion of dilation balloon (170) into the Eustachian tube (ET). As shown in FIG. 9, apart from inflating tip balloon (172) in vitro, as is depicted in FIG. 7A, tip balloon (172) may also or alternatively be inflated in vivo (i.e., in lieu of the state of operation shown in FIG. 7A or between the operation shown in FIG. 7A and the state of operation shown in FIG. 7B). Inflating in this order may be desirable in cases when the opening of the Eustachian tube (ET) is narrow enough to inhibit an inflated tip balloon (172) from passing through. Inflation of tip balloon (172) in vivo may be controlled by use of any of the features described above. Tip balloon (172) diameter may be determined through use of a pressure gauge or through visual observation with an endoscope (181) by using any of the examples previously described.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A catheter system comprising: a balloon dilation catheter having a proximal end portion and a distal end portion, the balloon dilation catheter comprising: an elongate shaft having a dilation balloon lumen, the dilation balloon lumen being configured to couple with a first fluid supply, an expandable dilation balloon coupled to the elongate shaft and fluidly connected to the dilation balloon lumen, the expandable dilation balloon being configured to transition between an inflated state and a non-inflated state, and an expandable stop element coupled to the elongate shaft, the expandable stop element being distal to the expandable dilation balloon, the expandable stop element being configured to selectively transition among a range of expanded states including a non-expanded state, a first expanded state, and a second expanded state, the expandable stop element defining an outer diameter such that the outer diameter defines a minimum diameter when the expandable stop element is in the non-expanded state and a maximum diameter when the expandable stop element is in the second expanded state.

### Example 2

The catheter system of Example 1, the expandable stop element being sized to fit within a Eustachian tube of a pediatric patient and configured to prevent translation beyond an isthmus of an adult patient when the outer diameter is greater than the minimum diameter.

### Example 3

The catheter system of any preceding Example, the expandable stop element being inflatable, the elongate shaft further including a stop element lumen in fluid communication with the expandable stop element.

### Example 4

The catheter system of any preceding Example, the stop element lumen being configured to couple with a second fluid supply.

### Example 5

The catheter system of any preceding Example, the stop element lumen being configured to couple with the first fluid supply.

### Example 6

The catheter system of any preceding Example, the stop element lumen including a pressure regulator configured to limit a fluid pressure at the expandable stop element.

### Example 7

The catheter system of any preceding Example, the pressure regulator including an indicia configured to indicate a maximum fluid pressure at the expandable stop element.

### Example 8

The catheter system of any preceding Example, the pressure regulator including an indicia configured to indicate the outer diameter of the expandable stop element.

### Example 9

The catheter system of any preceding Example, the stop element lumen including a check valve configured to prevent fluid flow from the expandable stop.

### Example 10

The catheter system of any preceding Example, the minimum diameter being less than or equal to an outer diameter of the elongate shaft.

### Example 11

The catheter system of any preceding Example, the elongate shaft distally terminating at the expandable stop element such that the expandable stop element forms a distal tip of the elongate shaft.

### Example 12

The catheter system of any preceding Example, further comprising: a handle configured to be gripped by an operator, the handle comprising: a guide port having a bore, the bore being configured to receive a proximal end portion of the balloon dilation catheter such that the balloon dilation catheter distally projects from the handle, and a balloon catheter movement actuator configured to translate the balloon dilation catheter longitudinally relative to the handle.

### Example 13

The catheter system of any preceding Example, further comprising: a guide catheter received within the bore of the guide port such that the guide catheter distally projects from the handle, the guide catheter being configured to slidably receive the balloon dilation catheter.

### Example 14

The catheter system of any preceding Example, the guide catheter being configured to engage a second fluid supply assembly and thereby direct fluid into the expandable stop element lumen to thereby inflate the expandable stop element to thereby increase the outer diameter to be greater than the minimum diameter.

### Example 15

The catheter system of any preceding Example, the expandable stop element being resiliently biased toward the non-expanded state.

### Example 16

The catheter system of any preceding Example, further including a distal tip gauge configured indicate the outer diameter to an operator.

### Example 17

The catheter system of any preceding Example, further including a valve in fluid communication with the expandable stop element, the valve being configured to selectively change the outer diameter.

### Example 18

A dilation catheter, comprising: an elongate shaft, the elongate shaft defining a first lumen and a second lumen, the second lumen being fluidly isolated from the first lumen; an expandable dilation balloon supported by the elongate shaft, the expandable dilation balloon being in fluid communication with the first lumen, the expandable dilation balloon being configured to transition between an inflated state and a non-inflated state; and an expandable tip balloon supported by the elongate shaft, the expandable tip balloon being distal to the expandable dilation balloon, the expandable tip balloon being in fluid communication with the second lumen, the expandable tip balloon being configured to selectively transition between an inflated state and a non-inflated state, the expandable tip balloon defining an outer diameter, the dilation catheter being configured such that the expandable tip balloon is capable of maintaining a state that is between the inflated state and the non-inflated state.

### Example 19

A method, the method comprising manufacturing the catheter system of any one of examples 1 through 17 such that the catheter system may be used in pediatric and adult patients.

### Example 20

The method of Example 19, the method further comprising maintaining the expandable stop element such that the outer diameter remains between the minimum and maximum diameters.

### Example 21

A balloon dilation catheter comprising: an elongate shaft; an expandable dilation balloon coupled to the elongate shaft and in fluid communication with a first fluid supply, the expandable dilation balloon being configured to transition between an inflated state and a non-inflated state, the inflated state being configured to dilate a Eustachian tube; and an expandable stop element coupled to the elongate shaft and in fluid communication with a second fluid supply, the expandable stop element being distal to the expandable dilation balloon, the expandable stop element being configured to selectively transition among a range of expanded states including a non-expanded state, a first expanded state, and a second expanded state through use of the second fluid supply.

### Example 22

A catheter system comprising a balloon dilation catheter having a proximal end portion and a distal end portion, the balloon dilation catheter comprising: a handle, an elongate shaft extending distally from the handle and including a shaft diameter, an expandable dilation balloon coupled to the elongate shaft and in fluid communication with a fluid supply, the expandable dilation balloon being configured to transition between an inflated state and a non-inflated state, and an expandable stop element coupled to the elongate shaft and in fluid communication with the fluid supply, the expandable stop element being distal to the expandable dilation balloon, the expandable stop element being configured to selectively transition among a range of expanded states including a non-expanded state, a first expanded state, and a second expanded state, the non-expanded state including a diameter that is smaller than the shaft diameter.

### Example 23

A method of using a balloon dilation catheter sized to dilate a Eustachian tube, the balloon dilation catheter including an expandable dilation balloon and an expandable stop element that is distal to the expandable dilation balloon, the expandable stop element being configured to selectively transition among a range of expanded states including a non-expanded state, a first expanded state, and a second expanded state, the first expanded state including a first diameter that is smaller than a second diameter of the second expanded state and to prevent advancement of the balloon dilation catheter beyond an isthmus of a Eustachian tube, the method comprising: selectively transitioning the expandable stop element from the non-expanded state to the first expanded state.

### Example 24

The method of Example 23, further comprising (i) gauging a size of the Eustachian tube, and (ii) selecting the first expanded state based on the gauged size of the Eustachian tube.

### V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A catheter system comprising:
a balloon dilation catheter having a proximal end portion and a distal end portion, the balloon dilation catheter comprising:
(i) an elongate shaft having a dilation balloon lumen, the dilation balloon lumen being configured to couple with a first fluid supply,
(ii) an expandable dilation balloon coupled to the elongate shaft and fluidly connected to the dilation balloon lumen, the expandable dilation balloon being configured to transition between an inflated state and a non-inflated state, and
(iii) an expandable stop element coupled to the elongate shaft, the expandable stop element being distal to the expandable dilation balloon, the expandable stop element being configured to selectively transition among a range of expanded states including a non-expanded state, a first expanded state, and a second expanded state,
the expandable stop element defining an outer diameter such that the outer diameter defines a minimum diameter when the expandable stop element is in the non-expanded state and a maximum diameter when the expandable stop element is in the second expanded state.

2. The catheter system of claim 1, the expandable stop element being sized to fit within a Eustachian tube of a pediatric patient and configured to prevent translation beyond an isthmus of an adult patient when the outer diameter is greater than the minimum diameter.

3. The catheter system of claim 1, the expandable stop element being inflatable, the elongate shaft further including a stop element lumen in fluid communication with the expandable stop element.

4. The catheter system of claim 1, the stop element lumen being configured to couple with a second fluid supply.

5. The catheter system of claim 1, the stop element lumen being configured to couple with the first fluid supply.

6. The catheter system of claim 1, the stop element lumen including a pressure regulator configured to limit a fluid pressure at the expandable stop element.

7. The catheter system of claim 1, the pressure regulator including an indicia configured to indicate a maximum fluid pressure at the expandable stop element.

8. The catheter system of claim 1, the pressure regulator including an indicia configured to indicate the outer diameter of the expandable stop element.

9. The catheter system of claim 1, the stop element lumen including a check valve configured to prevent fluid flow from the expandable stop.

10. The catheter system of claim 1, the minimum diameter being less than or equal to an outer diameter of the elongate shaft.

11. The catheter system of claim 1, the elongate shaft distally terminating at the expandable stop element such that the expandable stop element forms a distal tip of the elongate shaft.

12. The catheter system of claim 1, further comprising:
a handle configured to be gripped by an operator, the handle comprising:
(i) a guide port having a bore, the bore being configured to receive a proximal end portion of the balloon dilation catheter such that the balloon dilation catheter distally projects from the handle, and
(ii) a balloon catheter movement actuator configured to translate the balloon dilation catheter longitudinally relative to the handle.

13. The catheter system of claim 1, further comprising:
a guide catheter received within the bore of the guide port such that the guide catheter distally projects from the handle, the guide catheter being configured to slidably receive the balloon dilation catheter.

14. The catheter system of claim 1, the guide catheter being configured to engage a second fluid supply assembly and thereby direct fluid into the expandable stop element lumen to thereby inflate the expandable stop element to thereby increase the outer diameter to be greater than the minimum diameter.

15. The catheter system of claim 1, the expandable stop element being resiliently biased toward the non-expanded state.

16. The catheter system of claim 1, further including a distal tip gauge configured indicate the outer diameter to an operator.

17. The catheter system of claim 1, further including a valve in fluid communication with the expandable stop element, the valve being configured to selectively change the outer diameter.

18. A balloon dilation catheter comprising:
(a) an elongate shaft;
(b) an expandable dilation balloon coupled to the elongate shaft and in fluid communication with a first fluid supply, the expandable dilation balloon being configured to transition between an inflated state and a non-inflated state, the inflated state being configured to dilate a Eustachian tube; and
(c) an expandable stop element coupled to the elongate shaft and in fluid communication with a second fluid supply, the expandable stop element being distal to the expandable dilation balloon, the expandable stop element being configured to selectively transition among a range of expanded states including a non-expanded state, a first expanded state, and a second expanded state through use of the second fluid supply.

19. A method of using a balloon dilation catheter sized to dilate a Eustachian tube, the balloon dilation catheter including an expandable dilation balloon and an expandable stop element that is distal to the expandable dilation balloon, the expandable stop element being configured to selectively transition among a range of expanded states including a non-expanded state, a first expanded state, and a second expanded state, the first expanded state including a first diameter that is smaller than a second diameter of the second expanded state and to prevent advancement of the balloon dilation catheter beyond an isthmus of a Eustachian tube, the method comprising:
(i) selectively transitioning the expandable stop element from the non-expanded state to the first expanded state.

20. The method of claim 19, further comprising:
(i) gauging a size of the Eustachian tube, and
(ii) selecting the first expanded state based on the gauged size of the Eustachian tube.
